(19) United States
(12) Patent Application Publication
(10) Pub. No.: US 2020/0171167 A1
(43) Pub. Date: Jun. 4, 2020

(54) CATIONIC NUCLEIC ACID SCAVENGER AND USES THEREOF

(71) Applicant: THE TRUSTEES OF COLUMBIA UNIVERSITY IN THE CITY OF NEW YORK, New York, NY (US)

(72) Inventors: Kam W. LEONG, New York, NY (US); Wei LEONG, New York, NY (US); Bo PENG, New York, NY (US)

(21) Appl. No.: 16/618,281

(22) PCT Filed: May 24, 2018
(86) PCT No.: PCT/US2018/034305

§ 371 Date: Nov. 29, 2019

Related U.S. Application Data
(60) Provisional application No. 62/512,581, filed on May 30, 2017.

(51) Int. Cl. A61K 47/69; A61K 31/355; A61K 31/74; C07K 14/705; A61K 9/50

(52) U.S. Cl. CPC A61K 47/6929; A61K 31/355; A61K 31/74; B82Y 5/00; C07K 14/705; A61K 9/5031; A61K 47/6923

(57) ABSTRACT

The present invention discloses cationic nucleic acid scavengers to effectively inhibit the activation of multiple nucleic acid sensing pattern recognition receptors (PRRs) to treat an inflammatory or immune response which is induced by a nucleic acid through the activation of the PRRs. The cationic nucleic acid scavengers include water soluble cationic polymers, cationic nanoparticles, and cationic micro-particles, and bind the nucleic acid in a manner that is independent of the sequences, structure or chemistry of the nucleic acid.

This application claims the benefit of U.S. provisional application No. 62/512,581 filed May 30, 2017, the entire content of which is expressly incorporated herein by reference thereto.

### FIELD OF THE INVENTION

The present invention discloses cationic nucleic acid scavengers as anti-inflammatory agents to effectively inhibit the activation of multiple nucleic acid sensing pattern recognition receptors (PRRs). The cationic nucleic acid scavengers include water soluble cationic polymers, cationic nanoparticles, and cationic micro-particles.

### BACKGROUND OF THE INVENTION

The innate immune system, non-specific immune system, involves molecules, cells and complex mechanisms as self-protection to defend the body from harmful stimuli, such as damaged cells, irritants, pathogens or endogenous stress signals. Pattern recognition receptors (PRRs) allow immune cells, such as macrophages, neutrophils, natural killer cells and dendritic cells, to identify pathogen-associated molecular patterns (PAMPs) and damage-associated molecular patterns (DAMPs) derived from various harmful stimuli, such as exogenous products from infectious organisms or endogenous molecules from damaged cells.

For DAMPs to become recognizable patterns, it requires structural modifications, such as degradation, denaturation, post-translational modification and redox reactions occurred in the setting of cell death. DAMPs have highly diverse patterns, including small molecules and large molecules, such as ATP (adenosine triphosphate), uric acid, proteins, and nucleic acids (both DNA and RNA). (Pisetsky et al., Nucleic acid-binding polymers as anti-inflammatory agents: reducing the danger of nuclear attack, Expert Rev Clin Immunol, January 2012, 8(1) page 1-3)

During the invasion of pathogens, PRRs, such as TLRs (toll-like receptors), are activated to initiate intracellular signaling events that result in the expression of immune response genes including inflammatory and immune modulatory cytokines, chemokines, immune stimulatory receptors to kill pathogens and to initiate the process of developing acquired immunity (Takeda and Akira, Int. Immunol. 17:1-14, 2005; Akira et al, Cell 124:783-801, 2006).

Inappropriate or excessive activation of PRRs has been associated with the development of various inflammatory diseases, such as autoimmune diseases, transplant immunity and post-injury inflammation. Inappropriate activation of some members of the TLR family contribute to development of a variety of diseases including bacterial sepsis (TLR1, TLR2, TLR3, TLR4 and TLR9) (Wurfel et al, Am. J. Respir. Crit. Care Med. 178:710-720 (2008); Knuefermann et al, Circulation 110:3693-3698 (2004); Cavassani et al, J. Exp. Med. 205:2609-2621 (2008); Alves-Filho et al, Crit. Care Med. 34:461-470 (2006); Tsujimoto et al, J. Hepatol. 45:836-843 (2006)), non-infection systemic inflammatory response syndrome (TLR4) (Breslin et al, Shock 29:349-355 (2008)), multiple sclerosis (TLR3, TLR4 and TLR9) (Chen et al, Int. Immunopharmacol 7:1271-1285 (2007)), systemic lupus erythematosus (SLE) (TLR7 and TLR9) (Marshak-Rothstein and Rifkin, Annu. Rev. Immunol. 25:419-441 (2007)) and rheumatoid arthritis (TLR3, TLR4, TLR7, TLR8 and TLR9) (Choe et al, J. Exp. Med. 197:537-542 (2003); O'Neil, Nat. Clin. Pract. Rheumatol. 4:319-327 (2008)). Preclinical and clinical studies indicate that inhibition of TLR activity has therapeutic benefits for treating certain diseases. For example, diverse lipopolysaccharides-neutralizing agents and TLR4 antagonists have been evaluated to treat inflammatory diseases in animal and clinical studies (Leon et al, Pharm. Res. 25:1751-1761 (2008)). A TLR9 inhibitor, inhibitory CpG DNA (Plitas et al, J. Exp. Med. 205:1277-1283 (2008)), and an antagonistic anti-TLR3 antibody (Cavassani et al, J. Exp. Med. 205:2609-2621 (2008)) enhanced survival of a mouse with polymicrobial sepsis. Oligonucleotide-based TLR7 and TLR9 inhibitors prevented IFNα (interferon α) production from human plasmacytoid dendritic cells stimulated with serum from SLE patients (Barrat et al, J. Exp. Med. 202:1131-1139 (2005)).

Some PRRs function as nucleic acid sensors which are contributory to the inflammation responses. Pathological inflammatory responses can be induced by nucleic acids, originated from host cells or intracellular microorganisms. These nucleic acids are one type of DAMPs and can activate several cytoplasmic PRRs and nucleic acid-sensing TLRs including at least four different TLRs, i.e. TLR3, TLR7, TLR8 and TLR9. The inappropriate activation of these TLRs can cause pathological inflammatory responses resulting inflammatory and autoimmune diseases. (Lee et al., Nucleic acid-binding polymers as anti-inflammatory agents, Proc Natl Acad Sci USA, August 2011, 108(34) page 14055-14060)

TLRs are trans-membrane proteins comprising an extracellular domain of leucine-rich repeats and an intracellular toll/interleukin-1 receptor domain (Leulier and Lemaitre, Nat. Rev. Genet. 9:165-178, 2008). Several TLRs have been identified for recognizing particular molecular patterns. For example, TLR2, TLR4, TLR5, TLR6 and TLR11 recognize bacterial outer membrane molecules, such as lipopolysaccharide, peptidoglycan and lipoteic acid. TLR3, TLR7, TLR8 and TLR9 recognize bacterial, viral or endogenous nucleic acids (Kawai and Akira, Semin. Immunol. 19:24-32, 2007). TLRs locate in different cellular localizations, such as cell surface or endosome. TLR3, TLR7, TLR8 and TLR9 are localized mostly in endosomal compartments (Kawai and Akira, Semin. Immunol. 19:24-32, 2007). Interferon-inducible protein AIM2 (absent in melanoma 2) participates in inflammatory responses by contributing to the defense against bacterial and viral DNA, which is known to have two oligonucleotide-binding domains.

Some PRR inhibitors or specific TLR antagonists have shown effects in reducing inflammation. However, due to the blocking of PRR function, they could have adverse effects by preventing the immune system from responding as inflammatory defense. The inflammation caused by nucleic acids, such as activation of multiple nucleic acid sensing PRRs, can be treated with cationic materials that have strong nucleic acid binding affinity without blocking the function of PRRs. The cationic materials, i.e. the cationic nucleic acid scavengers, could remove the pathogenic nucleic acids to inhibit inflammation without interfering the normal cell immune functions. After the nucleic acid forms a complex with the cationic nucleic acid scavengers, the interaction between the nucleic acid and the nucleic acid sensing PRR is disrupted, thereby restricting inflammatory activities. The cationic nucleic acid scavengers can bind and neutralize nucleic acids independent of the sequences, structure or chemistry of the nucleic acids, thereby inhibiting the activation of multiple nucleic acid sensing PRRs without directly interacting with the nucleic acid sensing PRRs.

Sullenger et al. (WO 2014/169043 A1, Anti-inflammatory agents and methods of using the same, published Oct. 16, 2014) discloses methods of neutralizing the effects of pro-inflammatory nucleic acids by using cationic polymers and methods of identifying anti-inflammatory cationic polymers through screening combinatorial libraries of nucleic acid-binding polymers. Moreno et al. (Scavenging Damage and Pathogen Associated Molecules, Current Trends in Biomedical Engineering & Biosciences, vol 2, issue 1, March 2017) and Eppensteiner et al. (Immunothrombotic Activity of Damage-Associated Molecular Patterns and Extracellular Vesicles in Secondary Organ Failure Induced by Trauma and Sterile Insults, Front Immunol. 2018 Feb. 8; 9:190. doi: 10.3389/fimmu.2018.00190) discuss the use of nucleic acid-binding cationic polymers, such as polyamidoamine dendrimer, hexadimethrine bromide, and β-cyclodextrin-containing polymer to neutralize the ability of free DNA, RNA, and inorganic polyphosphate to activate nucleic acid-sensing TLRs and intrinsic blood coagulation cascade.

U.S. Pat. No. 9,468,650 B2 (Sullenger et al., Inhibition of endosomal toll-like receptor activation) discloses a method of inhibiting nucleic acid-induced activation of TLR3 or TLR9 to treat a inflammatory or immune response by administering to a patient a poly(amidoamine) (PAMAM) that binds a nucleic acid responsible for the induction of activation.

Despite the prior efforts, there is a need for compounds that can provide a similar function at lower toxicity. There also is a need for treating various inflammatory diseases, such as autoimmune diseases, transplant immunity and post-injury inflammation with such lower toxicity compounds. The present invention now addresses these needs and provides viable improvements that have not been previously disclosed in the art.

### SUMMARY OF THE INVENTION

The present invention now provides cationic nucleic acid scavengers to effectively inhibit the activation of multiple nucleic acid sensing pattern recognition receptors (PRRs) to treat an inflammatory or immune response which is induced by a nucleic acid through the activation of the PRRs. These scavengers are less toxic than others that are known in the art.

The present invention also provides a method of inhibiting activation of a pattern recognition receptor (PRR) to treat an inflammatory or immune response which is induced by the PRR which comprises administering to a patient in need thereof a scavenging agent comprising a cationic nucleic acid polymer in an amount and under conditions such that the inhibition of the activation is effected, wherein the PRR is activated by a nucleic acid and the agent binds the nucleic acid. Preferably, the PRR is a cytoplasmic PRR or a TLR. In one embodiment, the PRR is TLR3, TLR7, TLR8, TLR9 or AIM2 (absent in melanoma 2). In the present method, the agent binds the nucleic acid in a manner that is independent of the sequences, structure or chemistry of the nucleic acid and the agent is one of the water soluble cationic polymers, cationic nanoparticles, or cationic micro-particles disclosed herein.

In a preferred embodiment, the agent is a dendronized polymer which comprises polyester backbones and dendritic cationic side chains, wherein the polyester backbone comprises poly (alpha-bromo-3-caprolactone), wherein the dendritic cationic side chain comprises a propargyl core and dendritic polyamidoamine side chains. In another embodiment, the agent is a polydopamine-laponite. The agent may comprise poly(ε-caprolactone)-block-poly[2-(dimethylamino)ethyl methacrylate] (PCL-b-PDMAEMA) block copolymers. These agents can be provided in the form of a microparticle or platelet.

Advantageously, the present method further comprises the step of exposing the patient to a nucleic acid prior to administering the agent. Generally, the patient was already exposed to a nucleic acid prior to administering of the agent.

Typically, the nucleic acid is pathogen-derived or is released from dead or damaged cells of the patient. The method further comprises detecting inhibition of activation of TLR3 or TLR9 by measuring TNF-α (tumor necrosis factor-α) or IL-6 (Interleukin 6) production in the patient, detecting inhibition of activation of TLR3 or TLR9 by measuring the levels of TNF-α or IFN-α, detecting inhibition of activation of TLR3 or TLR9 using reporter cells (involving poly (I:C) or CpG), or detecting inhibition of activation of AIM2 by measuring IL-1βproduction or caspase 1 p20 expression.

The administration of the agent results in a reduction in the acute inflammatory response in the patient and the agent does not affect lipopolysaccharide-mediated inflammation. The administration of the agent can be used to treat patients suffering from a disease selected from the group consisting of rheumatoid arthritis, spinal cord injury, psoriasis, systemic lupus erythematosus, inflammatory bowel disease, traumatic brain injury, an infectious disease, a cardiovascular disease, cancer bacterial sepsis, multiple sclerosis, chronic obstructive pulmonary disease, and obesity.

The present method also can prevent or inhibit progression of a thrombotic disorder by administering to a patient in need thereof one of the agents disclosed herein, wherein the agent binds a nucleic acid responsible for the induction or progression of the disease. The agent is administered in an amount and under conditions such that the prevention or inhibition is affected. The details of the preferred embodiments of the present invention are set forth in the accompanying figures and detailed description herein. Once these details of the invention are known, numerous additional innovations and changes will become obvious and implementable to one of ordinary skill in the art.

### BRIEF DESCRIPTION OF THE FIGURES

Further features of the inventive concept, its nature and various advantages will be more apparent from the following detailed description, taken in conjunction with the accompanying figures:

FIG. 1 shows (A). Cell viability of RAW264.7 murine macrophages using PCL-g-PAMAM tested by MTT array. (B). TLR9 inhibition of PCL-g-PAMAM tested by Ramos-Blue™ reporter cell. (C) and (D). The activity of PCL-g-PAMAM in suppressing the TNF-α or IFN-α secretion of synovial fluid-derived primary macrophages.

FIG. 2. (A). Images show the evolution of the fluorescent signals of PCL-g-PAMAM throughout the CIA model rats. (B) and (C). Images show the distribution of fluorescent signals of PCL-g-PAMAM in the organs. (B) 2 h and (C) 9 h after injection. 1-Thymus; 2-Heart; 3-Lung; 4-Liver; 5 and 6-Kidney; 7-Pancreas; 8-Spleen; 9-Bladder; 10 and 11-Forepaw; 12 and 13-Hindpaw.

FIG. 3 shows in vivo anti-inflammatory activity of the cationic dendronized polymers in the CIA model rats. (a). Schematic depiction of experimental timeline to analyze the anti-inflammatory activity of PCL-g-PAMAM. (b). Average disease scores of RA model rats, 384-G2 (PCL-g-PAMAM-1) and 384-G3 (PCL-g-PAMAM-2) treated rats (n=6-9), 13 days after first immunization, both polymers were injected daily at 20 mg/kg by intravenous injection. (c). Micro-CT images of the ankle joints of left hind paws of the control group, CIA model group, 384-G3 and 384-G2 treated group. (d). H&E staining images of the ankle joints of left hind paws of the control group, CIA model group, 384-G3 and 384-G2 treated group.

FIG. 4. (A) The zeta potential, (B) diameters of laponite (La), dopamine modified laponite (La-DA), PEI grafted laponite (La-DA-PEI), (C) cell viability using La-DA-PEI tested by MTT array on mouse embryonic fibroblasts.

FIG. 5. Upper panel: (A) TLR9, (B) TLR3 inhibition of La-DA-PEI tested by Ramos-Blue™ reporter cells. Lower panel: AIM2 activation of normal human primary keratinocytes by poly A:T and genome DNA and AIM2 inhibition by PCL-g-PAMAM.

FIG. 6. TLR9 inhibition of La-DA-PEI tested by Ramos-Blue™ reporter cell.

FIG. 7. (A) Cell viability using PCL-b-PDMAEMA block copolymer micro-particles (MP) tested by MTT array on mouse embryonic fibroblasts. (B) TLR9 inhibition of MP tested by Ramos-Blue™ reporter cell.

FIG. 8. (A) DNA level in the supernatant of human astrocyte and microglia exposed to 50 μM H₂O₂ for 24 h with different concentrations of MP tested by PicoGreen array. (B) TNF-α level in the supernatant of human astrocyte and microglia exposed to 50 μM H₂O₂ for 24 h with different concentration of MP tested by ELISA array.

### DETAILED DESCRIPTION OF THE INVENTION

Throughout this description, the preferred embodiments and examples provided herein should be considered as exemplar, rather than as limitations of the present invention.

The invention now provides new cationic nucleic acid scavengers, including water soluble cationic polymers, cationic nanoparticles, and cationic micro-particles, as anti-inflammatory agents, preferably in pharmaceutical acceptable compositions. These agents are improvements over those disclosed in U.S. Pat. No. 9,468,650 B2 such that that patent is incorporated herein by reference in its entirety for its disclosure of related and common features.

The inflammation caused by nucleic acids, such as activation of multiple nucleic acid sensing PRRs, can now be treated with cationic materials that have strong nucleic acid binding affinity and low toxicity. The present invention provides a more effective and safe method to treat the inflammation caused by the pathogenic nucleic acids. The cationic materials, i.e. the cationic nucleic acid scavengers, remove the pathogenic nucleic acids to inhibit inflammation without interfering the normal cell immune functions.

The three types of cationic nucleic acid scavengers disclose herein can efficiently inhibit the activation of an inflammatory response against the nucleic acids for treating various inflammatory diseases. The common PRR inhibitors may result in immune suppression and increased risk of infection, since the common PRR inhibitors can completely block the normal function of specific PRRs. The redundancy of the nucleic acid sensors in the cells could also greatly compromise the therapeutic effect of the typical PRR inhibitors. The nucleic acid scavengers can prevent the simultaneous activation of multiple nucleic acid sensing PRRs without interfering the normal immune functions of the cells. Therefore, the cationic nucleic acid scavengers disclosed herein can be used as anti-inflammation agents by targeting the inflammatory response induced by nucleic acids in autoimmune diseases, transplant immunity, and post-injury inflammation, such as anti-inflammation agents to treat rheumatoid arthritis, spinal cord injury, psoriasis, systemic lupus erythematosus, inflammatory bowel disease and traumatic brain injury.

PRRs are pivotal components of immune systems to provide self-protection in defending the body from harmful stimuli, such as pathogens and damaged cells. A variety of PRRs, including RIG-I-like receptors (RLRs), dsRNA-dependent protein kinase R (PKR), DNA-dependent activator of IRFs (DAI) and TLRs can recognize diverse products of pathogens and damaged cells that are referred as PAMPs and DAMPs (Lotze et al, Immunol. Reviews 220:60-81 (2007)). TLRs play a central role in host innate and acquired immunity, as well as in the pathogenesis of various diseases, including infectious diseases, inflammatory diseases and autoimmune diseases. TLRs 3, 7, 8 and 9 are localized in endosomes and can be activated by microbial and host nucleic acids. AIM2 (absent in melanoma 2) participates in inflammatory responses by contributing to the defense against bacterial and viral DNA.

The water soluble cationic polymers of the present invention are a series of water soluble dendronized polymers which comprise polyester backbones and dendritic cationic side chains. The polyester backbone of the water soluble dendronized polymer is synthesized by azidation of poly (alpha-bromo-3-caprolactone). The dendritic cationic side chains of the water soluble dendronized polymers are dendritic polyamidoamine side chains containing a propargyl core. The dendronized polymers have high binding affinity to nucleic acids and low cytotoxicity, which can be used as anti-inflammatory agents providing the advantages in scavenging nucleic acids, such as agents to effectively inhibit the activation of multiple nucleic acid sensors. In one embodiment, the water soluble dendronized polymers of the present invention can effectively inhibit the activation of multiple nucleic acid sensing PRRs, including TLR3, TLR9 and AIM2 in B cells, human primary keratinocytes and human embryonic kidney 293 cells.

The cationic nanoparticle of the present invention is a polycation-grafted nanoparticle, including a cationic polydopamine-gafted laponite, which has a large surface area and a high charge density providing the advantages in scavenging nucleic acids. Laponite is a synthetic layered silicate similar to a synthetic smectic clay, which can be degraded into nontoxic products at neutral pH. The cationic nanoparticle of the present invention have high binding affinity to nucleic acids, which can be used as anti-inflammatory agents providing the advantages in scavenging nucleic acids, such as agents to effectively inhibit the activation of multiple nucleic acid sensing PRRs. In one embodiment, the cationic polydopamine-grafted laponite can effectively inhibit the activation of B cells induced by pathogenic nucleic acids.

The cationic micro-particles of the present invention are block copolymers which are generated by the self-assembly of poly(ε-caprolactone)-block-poly[2-(dimethylamino)ethyl methacrylate] (PCL-b-PDMAEMA). The relatively large size of the micro-particles prevents the cationic micro-particles of the present invention from being internalized by the immune cells. Therefore, the cationic micro-particles of the present invention provide advantages for continuously scavenging the nucleic acids at the inflammatory site without being internalized by the surrounding cells. In particular, the cationic micro-particles of the present invention provide the advantages of higher charge density and lower systemic toxicity, which are advantageous in reducing the rapid, widespread immune activation involving the recruitment of immune cells to the local injured site. The cationic micro-particles of the present invention have high binding affinity to nucleic acids, which can be used as anti-inflammatory agents providing the advantages in scavenging nucleic acids, such as agents to effectively inhibit the activation of multiple nucleic acid sensing PRRs. In one embodiment, the cationic micro-particles of the present invention can effectively inhibit the activation of B cells by the pathogenic nucleic acid and the activation of microglias by H₂O₂ induced cell damage. In addition, the cationic micro-particles do not exhibit significant in vivo toxicity up to 200 mg/kg in mice either with intra-peritoneal injection, subcutaneous injection or oral delivery.

Advantageously, the binding affinity of a cationic nucleic acid scavenger of the present invention for a nucleic acid, expressed in terms of Kd, is in the pM to μM range, preferably, less than or equal to 3×10⁸M⁻¹; expressed in terms of binding constant (K), the binding affinity is advantageously equal to or greater than 7×10⁸M⁻¹, preferably equal to or greater than 5×10⁹M⁻¹. Thus, the binding affinity of the sequence-independent cationic nucleic acid scavengers can be, for example, about 7×10⁹M⁻¹. “K” and “Kd” can be determined by methods known in the art, including surface plasmon resonance, a real time binding assay such as Biacore, or Isothermal Titration Calorimetry (ITC).

Preferred cationic nucleic acid scavengers of the present invention simultaneously limit the activation of multiple PRRs, including TLR3, TLR9 and AIM2.

The present invention also provides a method of controlling (inhibiting or preventing) autoimmune and/or inflammatory responses associated with activation of multiple PRRs, including TLR3, TLR9 and AIM2. Such responses play a role in the pathogenesis of diseases/disorders that are associated with presence in the circulation of the patient of free nucleic acids, either pathogen-derived (e.g., viral- or bacterial-derived) nucleic acids or nucleic acids released from dead or damaged host cells. Specific diseases/disorders that can be treated using the cationic nucleic acid scavengers of the present invention include rheumatoid arthritis, spinal cord injury, psoriasis, systemic lupus erythematosus, inflammatory bowel disease, traumatic brain injury, an infectious disease, a cardiovascular disease, cancer bacterial sepsis, multiple sclerosis, chronic obstructive pulmonary disease, and obesity.

Another invention of the cationic nucleic acid scavengers of the present invention is to counteract the effects of DNA and RNA molecules that are released from cells and subsequently induce thrombosis (Kannemeier et al, Proc. Natl. Acad. Sci. 104:6388-6393 (2007); Fuchs et al, Proc. Natl. Aad. Sci. Published Online before Print Aug. 23, 2010). Since the cationic nucleic acid scavengers described herein can bind RNA and DNA molecules and shield them from other potential binding partners, such nucleic acid scavengers can be employed to inhibit the ability of DNA and RNA molecules to bind and activate coagulation factors and platelets to limit nucleic acid-induced pathological blood coagulation. Thus the nucleic acid scavengers described herein represent novel entities for preventing the induction and progression of a variety of thrombotic disorders including myocardial infarction, stroke and deep vein thrombosis.

The cationic nucleic acid scavengers of the present invention, or pharmaceutically acceptable salts thereof, can be administered to the patient via any route such that effective levels are achieved in, for example, the bloodstream. The optimum dosing regimen will depend, for example, on the cationic nucleic acid scavenger, the patient and the effect sought. Typically, the cationic nucleic acid scavenger will be administered orally, transdermally, IV, IM, IP or SC. The cationic nucleic acid scavenger can also be administered, for example, directly to a target site, for example, directly to a tumor (e.g., a brain tumor) when cancer is the disease to be treated. Advantageously, the nucleic acid binding agent is administered as soon as clinical symptoms appear and administration is repeated as needed.

The cationic nucleic acid scavengers of the present invention, or pharmaceutically acceptable salts thereof, can be formulated with a carrier, diluent or excipient to yield a pharmaceutical composition. The precise nature of the compositions of the invention will depend, at least in part, on the nature of the nucleic acid binding agent and the route of administration. Optimum dosing regimens can be readily established by one skilled in the art and can vary with the nucleic acid binding agent, the patient and the effect sought.

It will be appreciated that the treatment methods of the present invention are useful in the fields of both human medicine and veterinary medicine. Thus, the patient (subject) to be treated can be a mammal preferably a human. For veterinary purposes the subject can be, for example, a farm animal such as a cow, pig, horse, goat or sheep, or a companion animal such as a dog or a cat.

The present invention also relates to methods of identifying nucleic acid binding agents suitable for use in the above-described methods, comprising: i) culturing PRR-containing cells with a first PRR agonist in the presence and absence of a test agent, ii) obtaining a supernatant sample from the culture of step (i); iii) analyzing the sample for the presence of a product of an intracellular signaling event initiated by PRR activation, and; iv) repeating steps (i)-(iii) with second PRR agonist having a sequence, structure or chemistry different from that of the first agonist; wherein a test agent that inhibits PRR agonist activation in a manner independent of sequence, structure or chemistry of the PRR agonist used is a candidate nucleic acid binding agent.

### EXAMPLE

The following examples illustrate the benefits and advantages of the present invention.

### Example 1

### Generation of Water Soluble Dendronized Polymers

The water soluble cationic polymers of the present invention are a series of water soluble dendronized polymers which comprise polyester backbones and dendritic cationic side chains. The polyester backbone of the water soluble dendronized polymer was synthesized by azidation of poly (alpha-bromo-3-caprolactone) which was obtained by ring opening polymerization of alpha-bromo-3-caprolactone. The dendritic cationic side chains of the water soluble dendronized polymers were dendritic polyamidoamine side chains containing a propargyl core, which were synthesized through divergent synthesis method by assembling from a core and extending outward by a series of reactions. The water soluble dendronized polymers of the present invention were subsequently generated by click reaction between the synthesized polyester backbone and the synthesized dendritic cationic side chain. The degree of polymerization of poly (alpha-bromo-3-caprolactone) could be from 70 to 400. The PAMAM dendrimer side chain grafted onto the polymers could be from 50% to 100%. The generation of PAMAM could be from 1 to 3. The amino group in each repeat unite could be from 2 to 8.

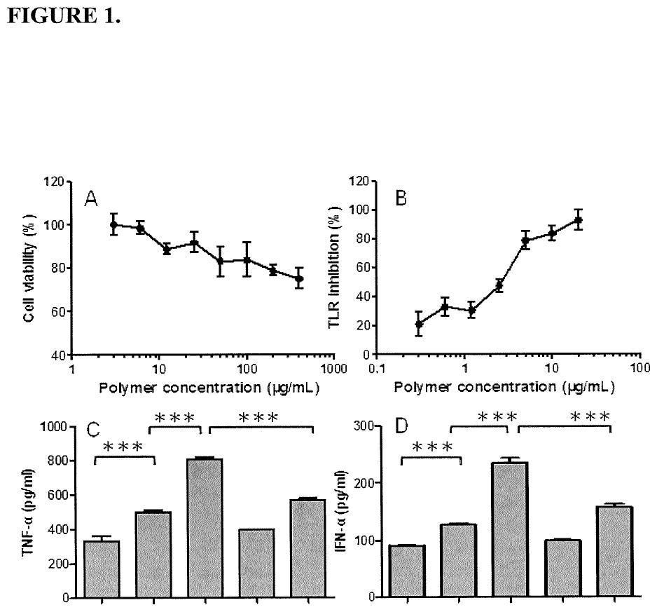

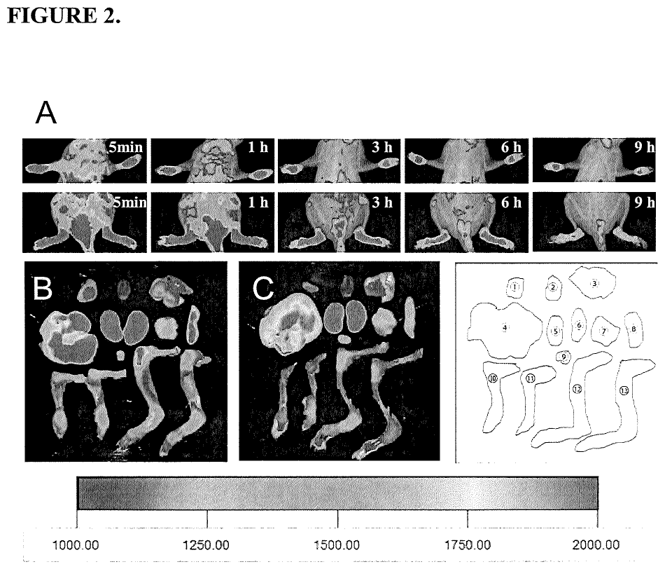

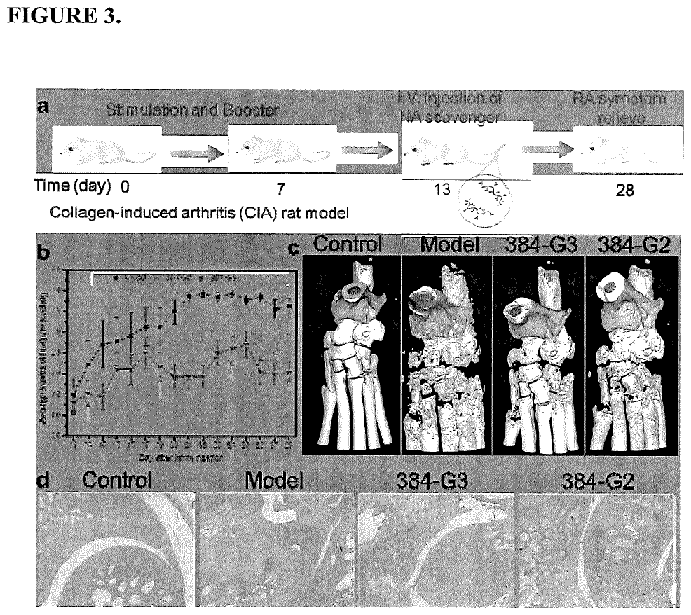

PCL-g-PAMAM (poly(ε-caprolactone)-grafted-poly(amidoamine)) was tested for its use to scavenge the nucleic acids and treat rheumatoid arthritis as shown in FIG. 1. FIG. 1-(A) shows cell viability of RAW264.7 murine macrophages using PCL-g-PAMAM, assayed by MTT (3-(4,5-dimethylthiazol-2-yl)-2,5-diphenyltetrazolium bromide) array. FIG. 1-(B) shows TLR9 inhibition of PCL-g-PAMAM tested by Ramos-Blue™ reporter cell (purchased from InvivoGen, Ramos-Blue™ cell is B lymphocyte cell lines that stably expresses an NF-κB/AP-1-inducible SEAP (secreted embryonic alkaline phosphatase) reporter gene). FIG. 1-(C) shows the activity of PCL-g-PAMAM in suppressing the TNF-α secretion of synovial fluid-derived primary macrophages. FIG. 1-(D) shows the activity of PCL-g-PAMAM in suppressing the IFN-α secretion of synovial fluid-derived primary macrophages. FIG. 2 shows the evolution of the fluorescent signals of PCL-g-PAMAM throughout the CIA (collagen-induced arthritis) model rats. The images in FIG. 2 show the distribution of fluorescent signals of PCL-g-PAMAM in different organs after injection at two time points, 2 and 9 hours. FIG. 3 shows the in vivo anti-inflammatory activity of the cationic dendronized polymers in the CIA model rats. Two of PCL-g-PAMAM polymers were used for testing the anti-inflammatory activity, i.e. 384-G2 (PCL-g-PAMAM-1, PCL384-g-PAMAM generation2) and 384-G3 (PCL-g-PAMAM-2, PCL384-g-PAMAM generation3). Both polymers were injected every day at dose of 20 mg/kg by intravenous injection.

### Example 2

### Generation of Polycation-Grafted Nanoparticles

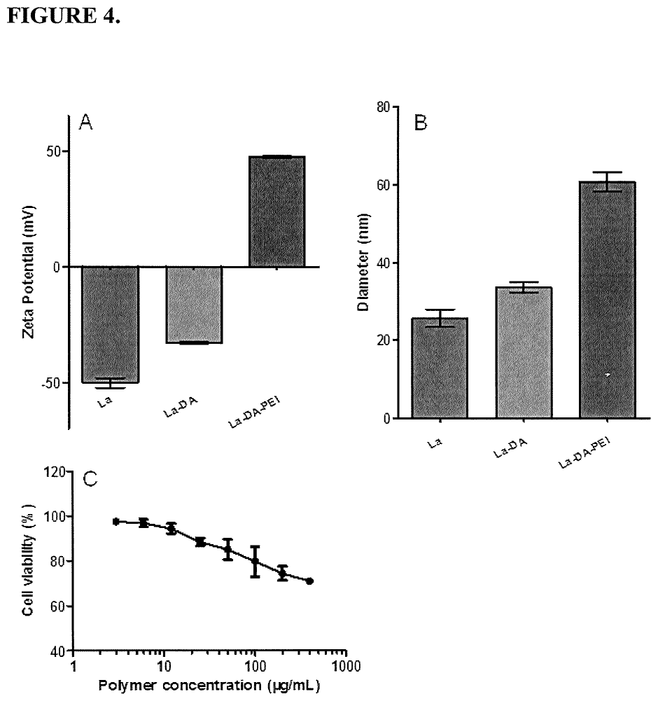

The cationic nanoparticle of the present invention is a polycation-grafted nanoparticle, including a cationic polydopamine-grafted laponite which was synthesized by surface modification of laponite mediated by dopamine. Laponite is a synthetic layered silicate similar to a synthetic smectic clay, which can be degraded into nontoxic products at neutral pH. In order to incorporate polydopamine to the surface of laponite, dopamines were added to a basic solution containing laponite. The cationic polydopamine-grafted laponite was generated by the Michael addition between polyethyleneimine (PEI) and polydopamine. The unreacted PEI and polydopamine were removed by centrifugation and washing repeatedly. After the modification, the zeta potential of the laponite significantly increased from −40 mV to 30 mV, indicating the modification of laponite. The laponite could be changed to any other inorganic particles including graphene, mesoporous silica nanoparticles, or Sepliolite. FIGS. 4A and 4B show the zeta potential and diameters of laponite (La), dopamine modified laponite (La-DA), and PEI grafted laponite (La-DA-PEI). FIG. 4C shows cell viability using La-DA-PEI tested by MTT array on mouse embryonic fibroblasts.

### Example 3

### Generation of Cationic Micro-Particles

The cationic micro-particles of the present invention are block copolymers which were synthesized by the self-assembly of poly(ε-caprolactone)-block-poly[2-(dimethylamino)ethyl methacrylate] (PCL-b-PDMAEMA). Methanol, a selective solvent of PDMAEMA, was gradually added into a THF (tetrahydrofuran) solution containing PCL-b-PDMAEMA while stirring. As soon as the addition of methanol was completed, the solution was kept still for at least 1 hour. Subsequently, the solution was dialyzed against water for 3 days. (Wang et al., A facile way to prepare crystalline platelets of block copolymers by crystallization-driven self-assembly, Polymer, 54(25), page 6760, 2013)

### Example 4

### Inhibition of the Activation of Nucleic Acid Sensors by Water Soluble Dendronized Polymers

The water soluble dendronized polymers prepared in example 1 can effectively inhibit the activation of multiple nucleic acid sensors, including TLR3, TLR9 and AIM2 in B cells, human primary keratinocytes and human embryonic kidney 293 cells.

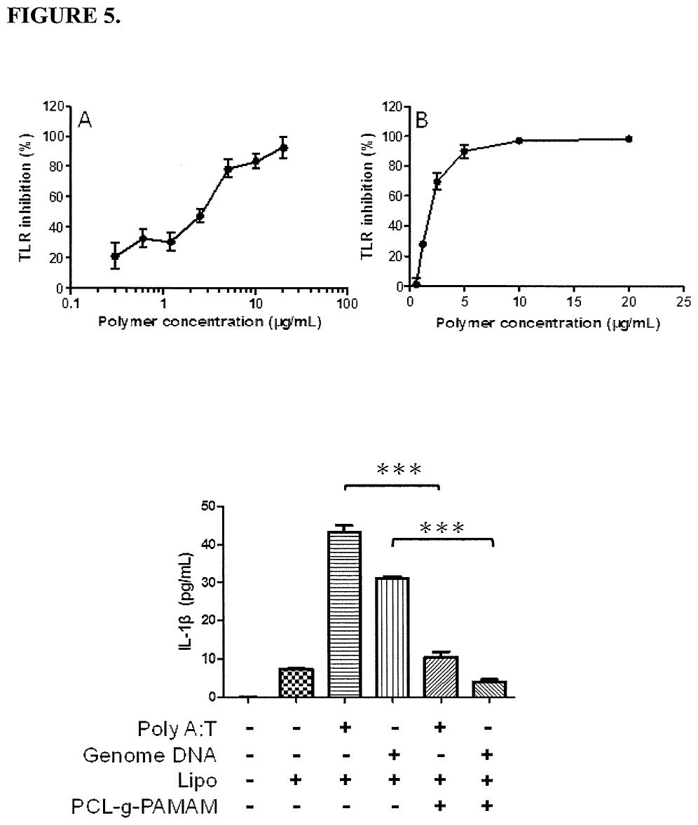

For TLR9 inhibition, 10 μL of 20 μg/mL of CpG ODN (CpG oligodeoxynucleotides, short single-stranded synthetic DNA molecules containing a cytosine triphosphate deoxynucleotide (“C”) followed by a guanine triphosphate deoxynucleotide (“G”); the “p” referring to the phosphordiester link between consecutive nucleotides) and 10 μL of polymer solution with different concentration were added into 180 μL Ramos-Blue™ cell suspension (2×10⁶ cells/mL) (Ramos-Blue™ cell is B cell) in the 96 well plates. In control group, PBS (phosphate-buffered saline) was used instead of polymer solution. After incubating the plates at 37° C., 5% CO₂ in the incubator for 24 h, 160 μL of QUANTI-Blue™ (purchased from InvivoGen, QUANTI-Blue™ is a colorimetric enzyme assay developed to determine any alkaline phosphatase activity (AP) in a biological sample, such as supernatants of cell cultures) was added into 40 μL of cell supernatant in the 96 well plates and incubated in 37° C. for 1.5 h. The secreted embryonic alkaline phosphatase (SEAP) level was determined by plate reader at 620 nm. For TLR3 inhibition, TLR3 agonist poly (I:C) (polyinosinic:polycytidylic acid) was used instead of CpG ODN. The upper panel of FIG. 5 shows TLR9 and TLR3 inhibition of La-DA-PEI, which was tested using Ramos-Blue™ reporter cells.

AIM2 activation was performed on normal human primary keratinocytes. The normal human primary keratinocytes were first primed by IFN-γ (100 ng/mL) and TNF-α (10 ng/ml) for 24 h. Then poly(dA:dT) or human genome DNA was transfected at 4 μg/ml with Lipofectamine RNAiMAX. Polymer was added simultaneously with poly (dA:dT) at 20 μg/ml. The activation of AIM2 was monitor by the level of IL-1β. IL-1β level was monitored by ELISA array. The lower panel of FIG. 5 shows AIM2 activation of normal human primary keratinocytes by poly A:T and genome DNA, and AIM2 inhibition by PCL-g-PAMAM.

### Example 5

### Inhibition of the Activation of Nucleic Acid Sensors by Cationic Polydopamine-Grafted Laponite

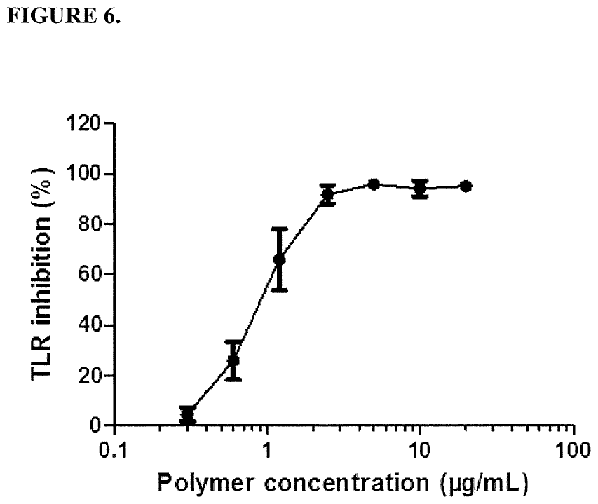

The cationic polydopamine-grafted laponite prepared in example 2 can effectively inhibit the activation of B cells induced by pathogenic nucleic acids. For TLR9 inhibition, 10 μL of CpG ODN (20 μg/mL) and 10 μL of cationic polydopamine-grafted laponite solution with different concentration were added into 180 μL Ramos-Blue™ cell suspension (2×10⁶ cells/mL) (Ramos-Blue™ cell is B cell) in the 96 well plates. In control group, PBS was used instead of polymer solution. After incubating the plates at 37° C., 5% CO₂ in the incubator for 24 h, 160 μL of QUANTI-Blue was added into 40 μL of cell supernatant in the 96 well plates and incubated in 37° C. for 1.5 h. The secreted embryonic alkaline phosphatase (SEAP) level was determined by plate reader at 620 nm. FIG. 6 shows TLR9 inhibition of La-DA-PEI tested by Ramos-Blue™ reporter cell.

### Example 6

### Inhibition of the Activation of Nucleic Acid Sensors by PCL-b-PDMAEMA Block Copolymer Micro-Particles

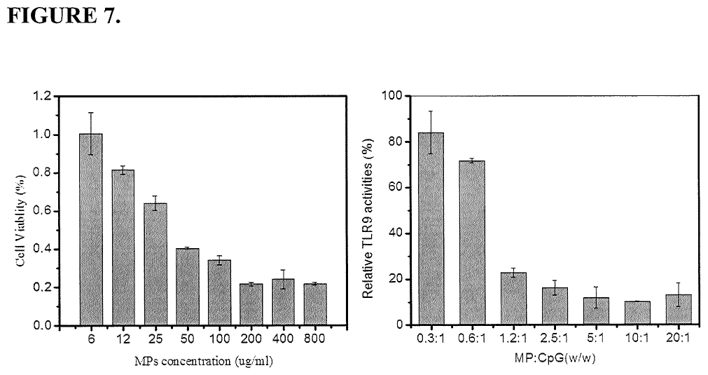

The PCL-b-PDMAEMA block copolymer micro-particles prepared in example 3 can effectively inhibit the activation of B cells by the pathogenic nucleic acid and the activation of microglias by H₂O₂ induced cell damage. TLR9 activation is tested using Ramos-Blue™ reporter cells as mentioned previously. FIG. 7 shows (A) Cell viability using PCL-b-PDMAEMA block copolymer micro-particles (MP) tested by MTT array on mouse embryonic fibroblasts. (B) TLR9 inhibition of MP tested by Ramos Blue™ reporter cell.

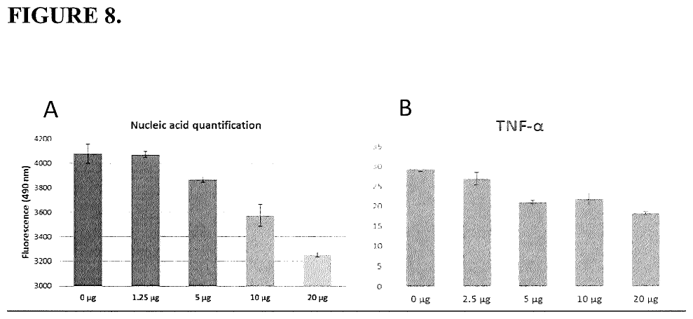

Regarding the activation of microglias by H₂O₂ induced cell damage, the human astrocyte and microglia was co-cultured and exposed to 50 μM H₂O₂ for 24 h. To inhibit the following NA-induced inflammation, MP in different concentration was added into the supernatant. The DNA and TNF-α level was monitored by PicoGreen array and ELISA array, respectively. FIG. 8 shows (A) DNA level in the supernatant of human astrocyte and microglia exposed to 50 μM H₂O₂ for 24 h with different concentration of MP by PicoGreen array. (B) TNF-α level in the supernatant of human astrocyte and microglia exposed to 50 μM H₂O₂ for 24 h with different concentration of MP by ELISA array.

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

What is claimed is:

1. A method of inhibiting an activation of a pattern recognition receptor (PRR) to treat an inflammatory or immune response which is induced by the PRR which comprises administering to a patient in need thereof an agent comprising a nucleic acid scavenger in an amount and under conditions such that the inhibition of the activation is effected, wherein the PRR is activated by a nucleic acid, wherein the agent binds the nucleic acid and is a selected from the group consisting of a dendronized polymer comprising a propargyl core and dendritic polyamidoamine side chains; a polydopamine-laponite; and a block copolymer that includes ε-caprolactone blocks.

2. The method of claim 1, wherein the PRR is a cytoplasmic PRR or a toll-like receptor (TLR).

3. The method of claim 1, wherein the PRR is TLR3, TLR7, TLR8, TLR9 or AIM2 (absent in melanoma 2).

4. The method of claim 1, wherein the agent binds the nucleic acid in a manner that is independent of the sequences, structure or chemistry of the nucleic acid.

5. The method of claim 1, wherein the agent is a water soluble cationic polymer, a cationic nanoparticle, or a cationic micro-particle.

6. The method of claim 1, wherein the agent is a dendronized polymer which comprises polyester backbones and dendritic cationic side chains, wherein the polyester backbone comprises poly (alpha-bromo-3-caprolactone), wherein the dendritic cationic side chain comprises a propargyl core and dendritic polyamidoamine side chains.

7. The method of claim 1, wherein the agent is a polydopamine-laponite.

8. The method of claim 1, wherein the agent comprises a poly(-caprolactone)-block-poly[2-(dimethylamino)ethyl methacrylate] (PCL-b-PDMAEMA) block copolymers.

9. The method of claim 1, wherein the agent is in the form of a microparticle or platelet.

10. The method of claim 1, wherein the patient was exposed to a nucleic acid prior to administering of the agent or further comprising exposing the patient to a nucleic acid prior to administering the agent.

11. The method of claim 1, wherein the nucleic acid is pathogen-derived or is released from dead or damaged cells of the patient.

12. The method of claim 3, further comprising detecting the inhibition of activation of TLR3 or TLR9 by measuring TNF-α or IL-6 production in the patient.

13. The method of claim 3, further comprising detecting the inhibition of activation of TLR3 or TLR9 by measuring the levels of TNF-α or IFN-α.

14. The method of claim 3, further comprising detecting the inhibition of activation of TLR3 or TLR9 using reporter cells involving poly (I:C) or CpG.

15. The method of claim 3, further comprising detecting the inhibition of activation of AIM2 by measuring IL-1β production or caspase 1 p20.

16. The method of claim 1, wherein administration of the agent results in a reduction in the acute inflammatory response in the patient.

17. The method of claim 1, wherein the agent does not affect lipopolysaccharide-mediated inflammation.

18. The method of claim 1, wherein the patient suffers from a disease selected from the group consisting of rheumatoid arthritis, spinal cord injury, psoriasis, systemic lupus erythematosus, inflammatory bowel disease, traumatic brain injury, an infectious disease, a cardiovascular disease, cancer bacterial sepsis, multiple sclerosis, chronic obstructive pulmonary disease, and obesity.

19. A method of preventing the induction of, or inhibiting the progression of, a thrombotic disorder comprising administering to a patient in need thereof an agent that binds a nucleic acid responsible for said induction or progression in an amount and under conditions such that the prevention or inhibition is effected, wherein the agent is a selected from the group consisting of a dendronized polymer comprising a propargyl core and dendritic polyamidoamine side chains; a polydopamine-laponite; and a block copolymer that includes ε-caprolactone blocks.
